(19) United States
(12) Patent Application Publication
(10) Pub. No.: US 2012/0172853 A1
(43) Pub. Date: Jul. 5, 2012

(54) Apparatus for ophthalmic laser surgery

(76) Inventors: Peter Riedel, Nurnberg (DE); Christof Donitzky, Eckental (DE); Klaus Vogler, Eckental (DE)

(21) Appl. No.: 13/395,917

(22) PCT Filed: Sep. 23, 2009
(86) PCT No.: PCT/EP09/06879

§ 371 Date: Mar. 14, 2012

(51) Int. Cl. A61F 9/009

(52) U.S. Cl. 606/4

(57) ABSTRACT

An apparatus for ophthalmic laser surgery comprises a contact surface for formative bearing contact of an eye to be treated, components for providing focussed, pulsed treatment laser radiation and for directing the same through the contact surface onto the eye, a measuring device for measuring the position of the contact surface along the direction of propagation of the treatment laser radiation, the measuring device providing position data representing the measured position of the contact surface at least one location of the contact surface, and an electronic process and control unit, which is connected to the measuring device and which is adapted to control the focus position of the treatment laser radiation in dependence on the position data. By measuring the position of the contact surface, the laser-surgery apparatus provides for compensation of unavoidable manufacturing tolerances of a contact element forming the contact surface, and thereby provides for precise referencing of the front surface of the eye in relation to the laser-surgery apparatus.

This application is a U.S. national phase application of co-pending international application number PCT/EP2009/006879 filed on Sep. 23, 2009, the disclosure of which is incorporated herein by reference.

### BACKGROUND

The invention relates to an apparatus for ophthalmic laser surgery.

### SUMMARY OF EXAMPLE EMBODIMENTS

Pulsed laser radiation is used in numerous techniques in the treatment of the human eye. In some of these techniques, the eye to be treated is pressed against a transparent contact element, which, with its contact surface that faces towards the eye, forms a reference surface for the positioning of the beam focus along the z direction (this, according to a usual notation, means the direction of propagation of the laser beam). In particular, treatment techniques used to produce cuts (incisions) in the eye tissue by means of focussed femtosecond laser radiation frequently employ such contact elements as a z reference for the laser focus. Owing to the contact element being pressed against the eye in such a way that the eye comes into close-fitting, flat bearing contact with the contact surface of the contact element that faces towards the eye, the contact element defines the z position of the front surface of the eye. Through referencing of the beam focus along the z direction in relation to this contact surface of the contact element, it is then ensured that the incision, or the individual photodisruption (the creation of an incision in the human eye by means of pulsed femtosecond laser radiation is normally based on the effect of so-called laser-induced optical breakdown, which results in a photodisruption) is located at the required position in the depth of the eye tissue.

Incisions made by a laser occur, for example, in the case of so-called Fs-LASIK, in which an anterior cover disc of the cornea, referred to as a flap in the art, is cut free by means of femtosecond laser radiation. As in the case of the classic LASIK technique (LASIK: laser in-situ keratomileusis), this flap, still hanging to the rest of the corneal tissue in a hinge region, can be folded aside in order to treat ablatively the underlying tissue by means of UV laser radiation. Another application for the making of intratissue incisions in the eye is that of so-called corneal lenticule extraction, in which, within the corneal tissue, a lens-shaped disk is cut out all round by means of femtosecond laser radiation. This disk is then removed through an additional incision extending to the eye surface (the additional incision is made either by means of a scalpel or likewise by means of femtosecond laser radiation). In the case of corneal transplants (keratoplasty), likewise, an incision can be made in the cornea by means of focussed, pulsed laser radiation.

For hygiene reasons, the contact element carrying the contact surface is often a disposable article, which has to be exchanged before each treatment. In the production of the contact elements, certain manufacturing tolerances cannot be precluded in general, even in the case of very high precision manufacturing. After an exchange of the contact element, therefore, the z position of the contact surface facing towards the eye can differ—even if only slightly—from that in the case of the previously used contact element. In the case of laser treatments by means of focussed femtosecond laser radiation, focus diameters are preferably as small as possible, in order to restrict the photodisruption as local as possible. Modern devices operate, for example, with focus diameters in the low one-digit μm range. A corresponding precision is desirable for incision guidance in the z direction. This requires a correspondingly precise manufacturing of the contact element, but this precision cannot always be ensured. In the case of reduced manufacturing precision of the contact element, this may result in an imprecise incision guidance along the z direction in the corneal tissue.

The object of the invention is to provide an apparatus for ophthalmic laser surgery that makes high-precision laser treatment of an eye possible.

To achieve this object, according to the invention, an apparatus for ophthalmic laser surgery is proposed, comprising a contact surface for formative bearing contact of an eye to be treated, components for providing focussed, pulsed treatment laser radiation and for directing the same through the contact surface onto the eye, a measuring device for measuring the position of the contact surface along the direction of propagation of the treatment laser radiation, the measuring device providing position data representing the measured position of the contact surface at least one location of the contact surface, and an electronic process and control unit, which is connected to the measuring device and which is adapted to control the focus position of the treatment laser radiation in dependence on the position data.

The invention makes it possible to determine and/or verify the position of the contact surface along the z direction (according to the direction of propagation of the treatment laser radiation) and to correct appropriate control parameters of the laser apparatus in dependence on the measured position of the contact surface. The z position of the contact surface is measured, for example, with reference to a given reference point in a fixed coordinate system of the laser-surgery apparatus. A differing z position of the contact surface in the coordinate system can be obtained for differing contact elements, depending on manufacturing precision. The process and control unit takes account of these variations in its control of the focus of the treatment laser radiation, such that an incision pattern or pattern of photodisruptions to be realized in the eye is actually located at the required location in the depth of the eye (i.e. at the required location in the z direction). In this way, highly precise incision depths are possible, for example, in the production of a LASIK flap, in the case of corneal lenticule extractions or in the case of keratoplasty procedures.

According to a development of the invention, the measuring device can be adapted to measure the position of the contact surface at a plurality of differing locations of the same. Through sampling of the contact surface at a plurality of locations of the same, it is possible, in addition to the determination of the z position of the contact surface, to acquire its angular orientation in space (angularity relative to the beam axis). This is because it cannot be precluded that the manufacturing tolerances mentioned also affect the relative angular orientation of the contact surface facing towards the eye relative to a predefined mounting surface of the contact element. Moreover, the manufacturing tolerances do not have to be equal all over in an x-y plane orthogonal to the z direction, for which reason multi-point sampling of the contact surface makes individual correction of the z position of the focus position possible for differing locations within the x-y plane.

The measuring device is preferably an optical coherence interferometric measuring device and for this purpose comprises an optical interferometer.

The contact surface is frequently part of an exchangeably arranged disposable component. It must be emphasized, of course, that the invention does not require any disposable nature of the element carrying the contact surface. The invention is equally applicable in the case of designs having a fixedly built-in, or at least multiple-use, contact surface.

The contact surface is preferably formed by a transparent applanation plate or a transparent contact glass. Applanation plates, at least on their plate side that faces towards the eye, have a planar applanation surface, by means of which levelling of the front side of the eye is achieved. The use of applanation plates for the purpose of referencing the eye to be treated may be advantageous in terms of a high beam quality of the laser radiation. Nevertheless, it is equally possible, within the scope of the invention, to use as a contact element a contact glass having a lens surface, facing towards the eye, that is typically concave or convex in form. The advantage of such contact glasses is, for example, a lesser increase of the pressure inside the eye upon pressing on the eye.

The contact surface is preferably formed by a transparent contact element that is part of a patient adapter, in particular exchangeably coupled to a focussing objective of the apparatus.

According to the invention there is further provided a method for laser treatment of an eye, comprising the steps:
producing a formative bearing contact between the eye and a contact surface,
providing focussed, pulsed treatment laser radiation and directing the same through the contact surface onto the eye,
generating position data representing a measured position of the contact surface at least one location of the contact surface along the direction of propagation of the treatment laser radiation, and
controlling the focus position of the treatment laser radiation in dependence on the generated position data.

In the case of the method, likewise, the position data can be representative of a measured position of the contact surface at a plurality of differing locations of the same.

### DETAILED DESCRIPTION OF THE DRAWING

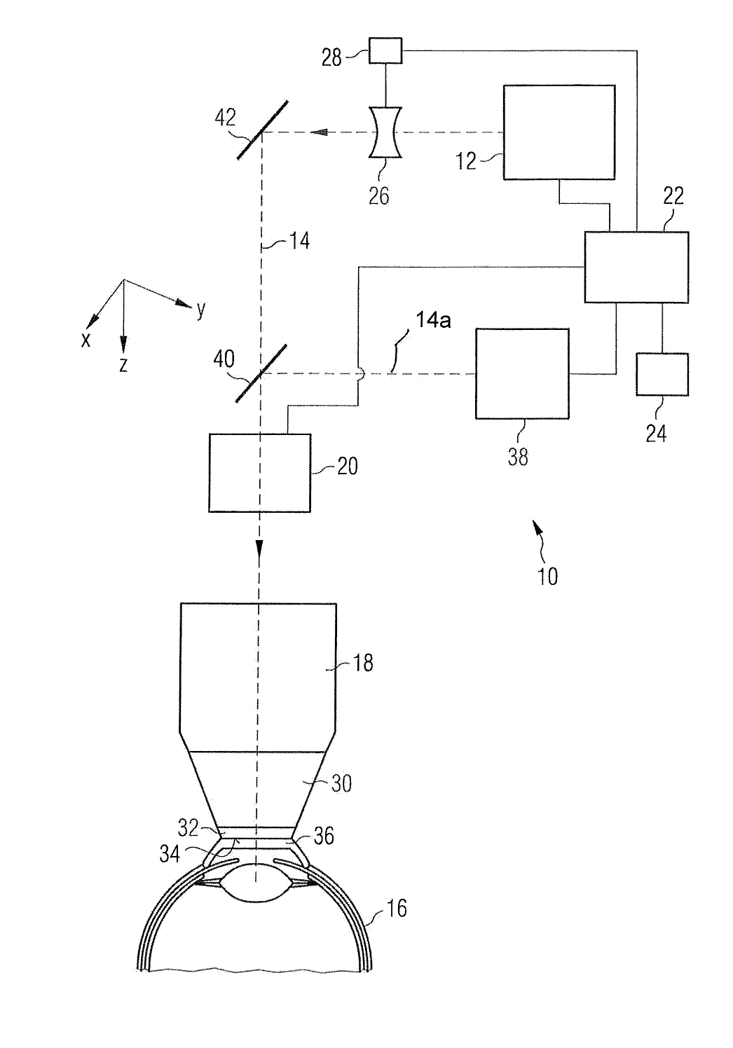

In the following, the invention is explained in further detail with reference to the single appended drawing. FIG. 1 shows, in a highly schematic form, an embodiment of an apparatus for ophthalmic laser surgery. The laser-surgery apparatus is denoted generally by 10. It comprises an fs laser 12, which emits pulsed laser radiation having pulse durations in the range of femtoseconds. The laser radiation propagates along an optical beam path 14, and finally reaches an eye 16 to be treated. Various components for guiding and shaping the laser radiation are arranged in the beam path 14. In particular, these components include a focussing objective 18 (for example, an F-Theta objective) and a scanner 20, which is connected upstream from the objective 18 and by means of which the laser radiation provided by the laser 12 can be deflected in a plane (x-y plane) orthogonal to the beam path 14. A coordinate system drawn in the FIGURE indicates this plane, and also a z axis defined by the direction of the beam path 14. The scanner 20 is constructed, for example, in a manner known per se, from a pair of galvanometrically controlled deflection mirrors, which are each responsible for deflecting the beam in the direction of one of the axes spanning the x-y plane. A central process and control unit 22 controls the scanner 20 in accordance with a control program that is stored in a memory 24 and that implements an incision profile to be generated in the eye 16 (the incision profile represented by a three-dimensional pattern of sampling points, at each of which a photodisruption is to be effected).

Furthermore, the mentioned components for guiding and shaping the laser radiation include at least one controllable optical element 26 for z adjustment of the beam focus of the laser radiation. In the example shown, this optical element is formed by a lens. An appropriate actuator 28, which is controlled by the process and control unit 22, serves to control the lens 26. For example, the lens 26 can be mechanically movable along the optical beam path 14. Alternatively, it is conceivable to use a controllable liquid lens of variable refractive power. In the case of an unchanged z position and also otherwise unchanged setting of the focussing objective 18, a z displacement of the beam focus can be achieved by moving of a longitudinally displaceable lens or by refractive index variation of a liquid lens. It is understood that other components, for instance a deformable mirror, are also conceivable for the purpose of z displacement of the beam focus. Owing to its comparatively higher inertia, it is expedient to set beam focus by the focussing objective 18 coarsely (i.e. focussing on a predefined z reference position) and to effect the z displacements of the beam focus that are predefined by the incision profile by a component arranged outside the focussing objective 18 and having a shorter reaction speed.

On the beam exit side, the focussing objective 18 is coupled to a patient adapter 30, which serves to produce a mechanical coupling between the eye 16 and the focussing objective 18. Usually, in the case of treatments of the type considered here, a suction ring, which is not represented in greater detail in the drawing but which is known per se, is placed onto the eye and fixed there by suction force. The suction ring and the patient adapter 30 form a defined mechanical interface that couples the patient adapter 30 to the suction ring. In this respect, reference can be made, for example, to the international patent application PCT/EP2008/006962, the entirety of which is hereby included by reference.

The patient adapter 30 serves as a carrier for a transparent contact element 32, which, in the example shown, is realized as a plane-parallel applanation plate. The patient adapter 30 comprises, for example, a taper sleeve body, the applanation plate 32 being arranged at its narrower (in the drawing, lower) sleeve end. In the region of the wider (in the drawing, upper) sleeve end, on the other hand, the patient adapter 30 is mounted on the focussing objective 18, where it has appropriate formations that, if required, enable the patient adapter 30 to be detachably fixed to the focussing objective 18.

Since it is in contact with the eye 16 during the treatment, the applanation plate 32 is an article that is critical from the aspect of hygiene, and which therefore, expediently, is to be exchanged after each treatment. For this purpose, the applanation plate 32 can be exchangeably mounted on the patient adapter 30. Alternatively, the patient adapter 30, together with the applanation plate 32, can form a disposable unit, for which purpose the applanation plate 32 can be non-detachably connected to the patient adapter 30.

In any case, the underside of the applanation plate 32 that faces towards the eye forms a planar contact surface 34, against which the eye 16 is pressed for the purpose of preparation of the treatment. This effects a levelling of the front surface of the eye while, at the same time, deforming the cornea of the eye 16, which is denoted by 36.

To enable the contact surface 34 to be used as a reference for the z control of the beam focus, it is necessary to know its z position in the coordinate system of the laser-surgery apparatus. Owing to unavoidable manufacturing tolerances, it cannot be precluded that, in the case of fitting of differing applanation plates or differing patient adapters 30 that are each equipped with an applanation plate 32, the z position and possibly also the angular orientation of the contact surface 34 exhibits variations of greater or lesser significance. Insofar as these variations are not taken into account in the z control of the beam focus, unwanted errors are obtained in the actual position of the incisions produced in the eye 16.

Consequently, the laser-surgery apparatus 10 includes an optical coherence interferometric measuring device 38, for example an OLCR measuring device (OLCR: optical low coherence reflectometry) that emits a measuring beam which, by means of an immovably arranged, semi-transparent deflection mirror 40, is coupled into the beam path 14 of the treatment laser radiation of the laser 12. The measuring device 38 brings the generated measuring beam into interference with a reflection beam coming back from the eye 16. The z position of the contact surface 34 can be determined with reference to the coordinate system of the laser-surgery apparatus from the interference measurement data obtained in this respect. For this reason, the interference measurement data can also be termed positional measurement data. The process and control unit 22 obtains the interference measurement data from the measuring device 38 and, from this data, calculates the z position of that location of the contact surface 34 at which the measuring beam impinged or through which the measuring beam passed. In the following laser treatment of the eye 16, the process and control unit 22 takes account of the thus determined actual z position of the contact surface 34 in the z control of the beam focus, this being in such a way that the incision is actually made at the intended position in the depth of the cornea 36. For this purpose, the z position of the beam focus that is to be set is referenced to the measured z position of the contact surface 34 by the process and control unit 22.

In the example shown, the measuring beam emitted by the measuring device 38 passes through the scanner 20. This enables the deflection function of the scanner 20 to be used also for the measuring beam. The scanner module 20 could also include a second, separate scanner, solely for the OLCR, which, being equipped with smaller mirrors, operates significantly more rapidly. However, the actual scanner mirror of the measuring device 38 can also be arranged separately in the first beam path 14a of the OLCR (not indicated in FIG. 1). Thus, a sampling of the contact surface 34 by the measuring beam and, consequently, a z measuring of the contact surface 34 at differing locations is possible. In this way, it is possible to generate a table or other suitable data structure that, for differing positions in the x-y plane, gives the z position of the contact surface 34 measured there in each case or gives a z correction value, which is calculated in dependence on the locally measured z position of the contact surface 34 and which is taken into account by the process and control unit 22 in the z control of the beam focus. If, for example, the incision profile is defined by a table that, for each photodisruption to be made, gives its z position with reference to a known, predetermined point in the coordinate system of the laser surgery apparatus, the table for the incision profile can be appropriately corrected by the process and control unit 22 on the basis of such z correction values.

In one embodiment, the scanner can include a pair of mirrors or a deflection unit operating according to another deflection technique, which is used jointly for the x-y deflection of the laser radiation and of the measuring beam. In another embodiment, the scanner 20 can include separate pairs of mirrors or, generally, separate deflection units, of which the one is used for the x-y deflection of the laser radiation and the other is used for the x-y deflection of the measuring beam. The deflection unit for the measuring beam could be equipped, for example, with smaller, more rapidly movable mirrors than the deflection unit for the laser radiation. In yet another embodiment, a deflection unit for the measuring beam can be arranged in that portion of the beam path of the measuring beam that is located in front of the deflection mirror 40. This portion is denoted by 14a in FIG. 1.

It is understood that, in yet an alternative embodiment, the scanner 20 can be located in front of the deflection mirror 40 in the direction of propagation of the laser radiation and, accordingly, a z measurement of the contact surface 34 at only a single location is possible. In this case, the process and control unit 22 can calculate a global z correction quantity which, in the z control of the beam focus, is applied equally for all sites in the x-y plane.

The reference 42 denotes a further immovable deflection mirror that serves to guide the treatment laser radiation.

1. Apparatus for ophthalmic laser surgery, comprising
a contact surface for formative bearing contact of an eye to be treated,
components for providing focussed, pulsed treatment laser radiation and for directing the same through the contact surface onto the eye,
a measuring device for measuring the position of the contact surface along the direction of propagation of the treatment laser radiation, the measuring device providing position data representing the measured position of the contact surface at least one location on the contact surface,
an electronic process and control unit, which is connected to the measuring device and which is adapted to control the focus position of the treatment laser radiation in dependence on the position data.

2. Apparatus according to claim 1, characterized in that the measuring device is adapted to measure the position of the contact surface at a plurality of differing locations of the contact surface.

3. Apparatus according to claim 1, characterized in that the measuring device comprises an optical interferometer.

4. Apparatus according to claim 1, characterized in that the contact surface is part of an exchangeably arranged disposable component.

5. Apparatus according to claim 1, characterized in that the contact surface is formed by a transparent applanation plate or a transparent contact glass.

6. Apparatus according to claim 1, characterized in that the contact surface is formed by a transparent contact element that is part of a patient adapter coupled to a focussing objective of the apparatus.

7. Apparatus according to claim 1, characterized in that the pulse duration of the treatment laser radiations is in the femtosecond range.

8. Method for laser treatment of an eye, comprising the steps:
producing a formative bearing contact between the eye and a contact surface,
providing focussed, pulsed treatment laser radiation and directing the same through the contact surface onto the eye,
generating position data representing a measured position of the contact surface at least one location of the contact surface along the direction of propagation of the treatment laser radiation,
controlling the focus position of the treatment laser radiation in dependence on the generated position data.

9. Method according to claim 8, the position data representing a measured position of the contact surface at a plurality of differing locations of the contact surface.
